(19) United States
(12) Patent Application Publication
(10) Pub. No.: US 2010/0227799 A1
(43) Pub. Date: Sep. 9, 2010

(54) SIMULTANEOUS PHOTODYNAMIC THERAPY AND PHOTO INDUCED POLYMERIZATION

(75) Inventor: JULIE TRUDEL, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/400,121

(22) Filed: Mar. 9, 2009

(51) Int. Cl. A61K 38/17; A61K 31/409

(52) U.S. Cl. 514/8; 514/185

(57) ABSTRACT

Described herein are methods and compositions for the combined treatments of photodynamic therapy and photo-induced polymerization. The wavelength of light needed to accomplish both therapies can be different or can be the same. In one embodiment, the photodynamic therapy is performed first and the photo-induced polymerization is performed thereafter. In other embodiments, both treatments are performed simultaneously.

### FIELD OF THE INVENTION

The present invention relates to methods and compositions for the combined treatments of photodynamic therapy and photo induced polymerization.

### BACKGROUND OF THE INVENTION

Polymers often contain matrices within their macrostructure networks. One type of polymeric matrix is a hydrogel, which can be defined as a water-containing polymeric network. Hydrogels have been beneficially used in medical settings such as, bioactive agent delivery, prevention of post-surgical adhesions, tissue repair, and the like.

Such hydrogels can be polymerized using light as an initiator. This method is commonly termed photo-induced polymerization and has been shown to be beneficial in such settings as vascular repair and regeneration. These hydrogels can even be polymerized in situ to ensure a proper polymer fit at the desired location and to reduce inflammation associated with implantable medical devices.

Another beneficial form of therapy involving light induction is photodynamic therapy. Such therapy is beneficial because it can provide reactive oxygen species for treatment of conditions in ophthalmology, oncology, and dermatology. Recent efforts have been made in treating regions of vasculature using photodynamic therapy, in particular, for the treatment of restenosis and plaque destabilization. In treating restenosis, the reactive oxygen species resulting from photodynamic therapy, inhibit smooth muscle cell proliferation without delaying regeneration of the affected tissue, thereby reducing healing time.

Effective methods wherein one or more photo-induced or photo-initiated therapies are utilized in treating a vascular condition would be beneficial and there is a need in the art for such methods. Methods such as these are described herein.

### SUMMARY OF THE INVENTION

Described herein are methods utilizing one or more photo-induced means to treat a vascular condition in need thereof. The combination of photodynamic therapy and photo-induced hydrogel polymerization can lead to improved efficacy in treating vascular conditions, increased acceptance of implantable medical devices and accelerated healing.

In one embodiment, a method of treating a vascular medical condition is described comprising the steps of: (a) selecting a surface to be treated; (b) providing at least one photosensitive molecule; (c) providing at least one photoinitiator; (d) delivering the at least one photosensitive molecule to a patient; (e) delivering the at least one photoinitiator to the surface to be treated; (f) delivering one or more polymerizable species to the surface to be treated; (g) providing a first light of an appropriate first wavelength to excite the photosensitive molecule at the surface to be treated, thereby forming a photodynamically treated surface; and (h) providing a second light of an appropriate second wavelength to excite the photoinitiator at the photodynamically treated surface initiating polymerization of the polymerizable species thereby forming a hydrogel over the photodynamically treated surface.

In another embodiment, the photosensitive molecule is at least one of a metal centered porphyrin molecule. In yet another embodiment, the photosensitive molecule is MV0611.

In still another embodiment, the photoinitiator is selected from the groups consisting of photosensitive dyes, quinones, hydroquinones, poly alkenes, polyaromatic compounds, ketones, unsaturated ketones, peroxides, halides, Eosin Y, Eosin B, flourone, erythrosine, flourecsein, indian yellow, derivatives thereof, and combinations thereof.

In other embodiments, the surface to be treated is selected from the groups consisting of a tissue surface, a tissue implant, a medical device and combinations thereof. In another embodiment, the at least one polymerizable species is a macromer formed from monomers selected from the groups consisting of methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, silicon oxides, polyethylene glycol, amide-containing monomers, isocyanide-containing monomers, derivatives and combinations thereof. In yet another embodiment, the at least one cross-linker is selected from the groups consisting of N-vinylpyrrolidone, polyethylene glycol derivatives, poly vinylpyrrolidinone, poly vinylpyrrolidinone derivatives, polyamides, polyurethanes, polysulfones, acrylates, derivatives and combinations thereof.

In some embodiments, the appropriate first wavelength is between about 400 nm and about 750 nm. In other embodiments, the appropriate second wavelength is between about 400 nm and about 750 nm. In further embodiments, the first appropriate wavelength and the second appropriate wavelength are the same and/or are provided at the same time. In another embodiment, the first appropriate wavelength and the second appropriate wavelength are provided sequentially. In still another embodiment, the first appropriate wavelength and the second appropriate wavelength are the same.

In another embodiment, the polymerizable species further comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, and wound healing proteins. In a further embodiment, the bioactive agent comprises at least one compound selected from the group consisting of sirolimus (rapamycin), tacrolimus (FK506), everolimus (certican), temsirolimus (CCI-779) and zotarolimus (ABT-578). In still a further embodiment, the wound healing proteins comprise tenascin, thrombospondin and osteopontin.

In another embodiment, the delivery of the at least one photosensitive molecule is by oral administration, intravenous administration or is delivered locally using a catheter.

In further embodiments, the at least one photosensitive molecule and the at least one photoinitiator are the same. In yet anther embodiment, the method further comprises the step of performing angioplasty to treat the vascular medical condition. In other embodiments, the method further comprises deploying a stent to treat the vascular medical condition.

Further described herein is a method of treating a vascular medical condition comprising the steps of: (a) selecting a vessel to be treated; (b) delivering MV0611 to a patient orally; (c) performing angioplasty; (d) deploying a stent into the vessel to be treated thereby forming a stented vessel; (e) delivering the eosin-Y to the stented vessel; (f) delivering a cross-linker and one or more macromer to the stented vessel; (g) providing a first light of an appropriate first wavelength to the stented vessel, thereby exciting MV0611 and forming a photodynamically treated vessel; (h) providing a second light of an appropriate second wavelength to excite the eosin-Y in the photodynamically treated vessel thereby initiating polymerization of the cross-linker and one or more macromer; and (i) forming a hydrogel over the photodynamically treated vessel.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the terpolymers of the present invention.

Proteins involved in wound healing are also within the scope of the term bioactive agent. Exemplary wound healing proteins include without limitation, tenascin, thrombospondin and osteopontin.

Another class of bioactive agents useful according to the present description include encapsulated small molecules. These molecules can be encapsulated in one or more delivery vehicle, for example, a liposome or a hydrogel coating or implant.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Cross-linking Agent: As used herein, “cross-linking agent” refers to a monomer that, when polymerized, covalently bonds one polymer chain to another.

Hydrogel: As used herein “hydrogel” refers to a water-containing polymer network.

Initiator: As used herein “initiator” refers to a molecule that initiates a polymerization reaction such as, but not limited to, an amino alcohol.

Macromer: As used herein “macromer” refers to a macromolecule, in particular a polymer, that can be further polymerized or cross-linked.

Photosensitive Molecule: As used herein “photosensitive molecule” refers to a molecule that becomes more reactive when exposed to light (photons). The term chromophore, as used herein, can also be sued to describe a photosensitive molecule. In both cases, the molecules absorb light of at least one particular wavelength, although typically a band of light, and move electrons from a ground state to an excited state.

### DETAILED DESCRIPTION OF THE INVENTION

Described herein are methods utilizing the combination of two or more light-induced (e.g. photo-induced or photo-initiated), therapies to treat a medical condition. The combination of photodynamic therapy and photo-induced hydrogel polymerization is an exemplary combination and can lead to improved efficacy in treating vascular conditions, increased acceptance of implantable medical devices and accelerated healing.

The first step in performing a method as described herein is to select a surface or area of a patient to be treated. The surface or area to be treated is selected from a tissue surface, such as a vessel or lumen wall, a tissue implant, a medical device or combinations thereof. The tissue implants are selected from organ transplants, cultured cells, cultured tissue, skin, bone, ligaments, blood vessels, and heart valves. The medical device is selected from joint implants, dental implants, soft tissue cosmetic prostheses, wound dressings, vascular prostheses, and ophthalmic prostheses. In an exemplary embodiment according to the present disclosure, the tissue surface is a stenosed vessel and the medical device is a vascular stent.

Once a surface or area of a patient to be treated has been identified, photodynamic therapy can be preformed in situ at an appropriate wavelength of light by exciting at least one photosensitive molecule. Photosensitive molecules can include metal centered porphyrin molecules such as those disclosed in U.S. Pat. No. 6,827,926 which is incorporated herein by reference for all it contains regarding porphyrin molecules. Excitation wavelengths of several photosensitive molecules are well known to persons of ordinary skill in the art. Excitation wavelengths of other molecules can easily be established by ascertaining spectral data for a given molecule and determining from that data a region of wavelengths responsible for reactive oxygen species generation.

In one embodiment according to the present description, the photosensitive molecule is gallium chloride mesoporphyrin dimethyl ester (MV0611) (Miravant Pharmaceuticals, Inc., Santa Barbara, Calif.). MV0611 has the following structure.

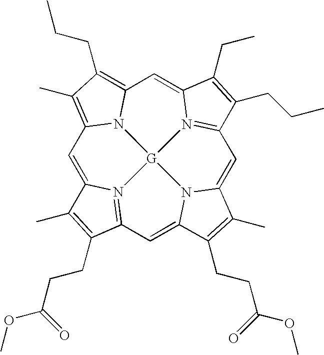

The photosensitive molecule can be delivered locally to a specific surface in need of treatment by delivery using a needle or catheter. The photosensitive molecule can also be introduced systemically by oral ingestion, subcutaneously, or intravenously. If administered orally, the photosensitive molecule can be present in a tablet or a liquid form; such oral forms are well known to those skilled in the art.

The photosensitive molecules described herein are substantially dormant in a hemodynamic environment and only become reactive upon excitation at a particular wavelength of light as previously established. Substantially dormant as used herein means that less than about 10% of the photosensitive molecules delivered to the hemodynamic environment react with the surrounding hemodynamic fluids before the first pass of the liver. In other embodiments, less than about 5%, more preferably 1% of the photosensitive molecules delivered to the hemodynamic environment react with the surrounding hemodynamic fluids before the first pass of the liver. After delivery, light at a pre-established wavelength can be directed at a particular region in need of therapy, thereby activating the photosensitive molecules.

Light can be directed to a particular region by several different means. For example, means for delivering light, if the region of treatment is a vessel undergoing restenosis can be provided during an angioplasty procedure, in a non-limiting example, via a catheter with a light emitting element thereon. Light can also be delivered via a non-invasive procedure, for example, by shining light outside the body in the general region of treatment, for example, over the entire thoracic region.

The photosensitive molecules themselves absorb light at one or more particular wavelengths and generate at least one reactive oxygen species. The particular wavelength or wavelengths of light are commonly referred to as excitation wavelengths. The excitation wavelength is highly dependent on the photosensitive molecule chosen for treatment, but is preferably in the visible spectrum of light, more preferably between about 400 nm and about 750 nm. In other embodiments, the wavelength is between about 500 nm and about 600 nm or between about 525 nm and about 550 nm. Most preferably, the excitation wavelengths are about 532 nm and about 490 nm. MV0611, for example, can absorb light at about 532 nm and at about 490 nm. This absorption can lead to the release of reactive oxygen species.

The reactive oxygen species generated by activation or excitation of the photosensitive molecule include superoxide radical, hydrogen peroxide, singlet oxygen and hydroxyl radical to name a few. The reactive oxygen species, once generated, can inhibit smooth muscle cell proliferation which is commonly responsible for intimal hyperplasia and various other vascular conditions known in the art.

The photodynamic therapy described herein is illustrated in the non-limiting uses in a hemofluidic environment, but can also be used in non-hemodynamic environments, such as, in the urinary tract or within cerebral spinal fluids.

The methods described herein further utilize photo-induced hydrogel formation, or photo-inducted polymerization, at a desired treatment site or area to achieve the benefits further described. Photo-induced hydrogel formation involves the formation of hydrogels by photoinitiating polymerization of one or more polymerizable species. Ploymerizable species include one or more cross-linker, one or more macromer, one or more monomer, one or more catalyst, or combinations thereof.

In one exemplary method, at least one photoinitiator and at least one cross-linking agent are delivered to a surface in need of treatment. The subsequent photo-induced polymerization involves exposing the photoinitiator and the cross-linking agent to light an appropriate wavelength of light thereby exciting the photoinitiator and in turn activating the cross-linker. This step activates the surface in need of treatment. Then, the activated surface is exposed to a mixture of one or more macromer and/or monomer, such that a hydrogel is formed on the activated surface.

The photoinitiators described herein can either be a molecular capable of exciting a cross-linking agent or can be an initiator capable of initiating polymerization upon excitation at an appropriate wavelength. Further, photoinitators described herein can be the same as, or similar to, the photosensitive molecules described herein used for photodynamic therapy.

Photoinitators suitable in the polymerization of hydrogels include, but are not limited to, photosensitive dyes, quinones, hydroquinones, polyalkenes, polyaromatic compounds, ketones, unsaturated ketones, peroxides, halides, Eosin Y, Eosin B, flourone, erythrosine, flourecsein, indian yellow, derivatives thereof, and/or combinations thereof. In one embodiment, the photosensitive molecule is Eosin Y. In another embodiment, the photosensitive molecule is Eosin B.

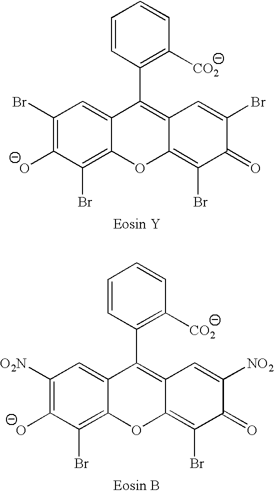

In another embodiment, the photosensitive molecule is fluorone.

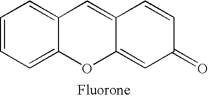

In yet another embodiment, the photosensitive molecule is erythrosine.

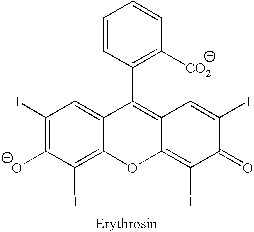

In still another embodiment, the photosensitive molecule is fluorescein.

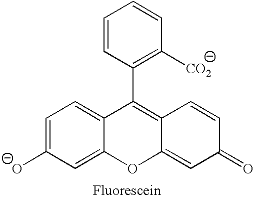

In even further embodiments, the photosensitive molecule is indian yellow.

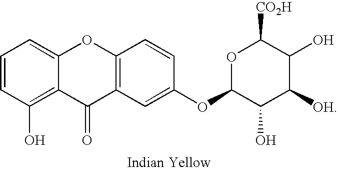

Each of the photoinitiators disclosed herein requires exposure to light of an appropriate wavelength to excite and activate the molecule. Each photoinitiator may have a different or similar excitation wavelength. For example, and not intended as a limitation, Eosin B is activated by light having wavelengths of about 511 nm to about 520 nm. In a further example, also not intended as a limitation, Eosin Y is activated by light having a wavelength of about 490 nm, but has a broad absorption spectra from about 410 nm to about 550 nm. Excitation wavelengths of photoinitiators are well known to persons of ordinary skill in the art.

Depending on the particular photoinitiator, the excitation wavelength is in the visible spectrum, preferably between about 400 nm and about 750 nm. More preferably, the wavelength is between about 500 nm and about 600 nm. In particular embodiments, the excitation wavelength is about 532 nm or about 490 nm. Excitation wavelengths in the ultraviolet range are also within the scope of the present description. The photoinitiators described herein generally absorb light in the ultraviolet range as a result of the high degree of resonance within the molecules.

Exemplary photoinitiators and photosensitive molecules, and their respective excitation wavelengths are described in U.S. Pat. No. 6,602,975 issued to Hubbell et al. which is hereby incorporated by reference for all it contains regarding photoinitiators and photosensitive molecules.

The macromers according to the present description are formed of one or more monomers. Monomers themselves can also be polymerized, either with or without macromers, and include, but are not limited to, methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, silicon oxides, polyethylene glycol, amide-containing monomers, isocyanide-containing monomers, derivatives thereof, and/or combinations thereof.

Light at an appropriate wavelength to excite a photoinitiator can be directed at a particular region or surface in need thereof. Light can be delivered in the same manner used for photodynamic therapy and can even be the same light source delivering light at the same wavelength. In other instances, the light can be delivered at a different time, by a different method, by a different source and/or at a different wavelength.

In some embodiments, the photoinitiators described herein can be used to further excite a second initiator which initiates polymerization of the hydrogels. Second initiators suitable for initiating the polymerization of hydrogels include amino alcohols such as, but not limited to, methyldiethanolamine, triethanolamine, thiols, C₁ to C₁₂ alkyls, C₃ to C₁₂ alkenyls, C₃ to C₁₂ alkynyls, C₆ to C₁₄ aryls, C₄ to C₁₂ heterocyc alkyls, C₄ to C₁₂ heterocyclic alkenyls, C₄ to C₁₂ heterocyclic aryls, derivatives thereof, and/or combinations thereof.

Cross-linking agents suitable for forming the hydrogels described herein include, but are not limited to, N-vinylpyrrolidone, polyethylene glycol derivatives, poly vinylpyrrolidinone, poly vinylpyrrolidinone derivatives, polyamides, polyurethanes, polysulfones, acrylates, derivatives thereof, and/or combinations thereof.

The hydrogels produced according to the present description can be utilized with or without a medical device. The hydrogels can be formed on medical devices in situ or can be formed directly on tissue in situ. In some instances, the hydrogels can be formed between the tissue and a medical device. Depending on the particular condition being treated, the hydrogels can be formed on selected portions of tissue, selected portions of a medical device or combinations thereof.

The hydrogels formed by photo-induced polymerization can further comprise at least one bioactive agent. The bioactive agents can be mixed with the hydrogel components before polymerization, and after polymerization, end up incorporated within the hydrogel. Exemplary, non limiting examples of bioactive agents that can be incorporated into the hydrogels include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers of the present invention.

Proteins involved in wound healing are also useful according to the present description as bioactive agents incorporated into the hydrogels. Exemplary proteins involved in wound healing include without limitation, tenascin, thrombospondin and osteopontin.

In one particular method according to the present description, a region of vasculature is first treated with photodynamic therapy wherein the photosensitive molecule is excited at a selected wavelength. Following the photodynamic therapy, a hydrogel can be formed in situ on at least a portion of the region previously treated with photodynamic therapy. The wavelength of the photo-initiation of the hydrogel polymerization is at a selected wavelength that is different that that used for photodynamic therapy. Using such photodynamic therapy allows for the treatment of vasculature in need thereof, and subsequent photo-induced hydrogel polymerization over the photodynamically treated vasculature aids in healing.

In another method, a region of vasculature is treated with photodynamic therapy, the photosensitive molecule is excited at a selected wavelength. Simultaneously, a hydrogel can be formed in situ on at least a portion of the region being treated with photodynamic therapy. The wavelength of the photo-initiation of the hydrogel polymerization is at a selected wavelength that is different that that used for photodynamic therapy.

In another method, the excitation wavelength of the photosensitive molecule for photodynamic therapy and the excitation wavelength for photo-initiation of polymerization can be the same.

Further, in methods described herein, the excitation of the photosensitive molecule for photodynamic therapy and the excitation for photo-initiation of polymerization can be done simultaneously.

Further still, in other methods described herein, the excitation of the photosensitive molecule for photodynamic therapy and the excitation for photo-initiation of polymerization can be done sequentially.

Even further still, the present methods can be utilized during, before or after angioplasty or stenting of a body lumen, for example a vessel. It is preferable that angioplasty take place before any therapy is administered to a vessel so that the angioplastied vessel can benefit from both the photodynamic therapy and the coating of a photo-induced polymerization. Both therapies will aid in healing of the vessel. It is preferable that if stenting is used to treat a vessel, the stent be deployed before the polymerization of the hydrogel so that the hydrogel can intertwine itself within the stent and into the adjacent vessel wall. Photodynamic therapy is preferable both before and after stenting of a vessel and is dependent on the severity and needs of the patient. For example, photodynamic therapy can precede stenting to treat the vessel prior to the stenting procedure. In contrast, photodynamic therapy can immediately follow stenting to treat the vessel after the stenting procedure in order to initiate healing after the stent has been deployed.

### EXAMPLE 1

### Method of Treating a Diseased Vessel

A patient takes an oral dose of liquid containing a photosensitive molecule. After a predetermined amount of time sufficient for the photosensitive molecule to fully distribute itself systemically in the patient's body, the patient can undergo angioplasty on a stenosed vessel. The angioplasty can thereby properly expand the vessel.

After angioplasty, eosin-Y is applied over the interior wall of the angioplastied vessel using a catheter. Thereafter, the polymerizable species are applied over the eosin-Y at the angioplastied lesion also using a catheter.

After eosin-Y administration, polymerizable species are applied at the angioplastied lesion, and the photosensitive molecule has had sufficient time to become systemically distributed though the patient's body, light at a wavelength of 532 nm can be applied to the angioplastied region via a light element at the end of a catheter. The light at 532 nm will activate the photosensitive molecule to provide photodynamic therapy at the site and will also simultaneously activate the eosin-Y which will initiate with polymerization of the polymerizable species at the site thereby forming a hydrogel.

### EXAMPLE 2

### Alternate Method of Treating a Diseased Vessel

A patient takes an oral dose of liquid containing MV0611. After a predetermined amount of time sufficient for the MV0611 to fully distribute itself systemically in the patient's body, the patient can undergo angioplasty. The angioplasty can thereby correct problems in the stenosed vessel.

After angioplasty, eosin-B is applied over the angioplastied lesion using a catheter. Thereafter the polymerizable species are applied over the eosin-B on the angioplastied vessel using a needle.

After eosin-B administration, polymerizable species are applied at the angioplastied lesion, and the photosensitizing agent has had sufficient time to become systemically distributed though the patient's body, light at a wavelength of 532 nm can be applied to the angioplastied region. Light at 532 nm will active the photosensitizing agent to provide therapy at the site. Thereafter, light at 515 nm can be applied at the site thereby activating the eosin-B which will initiate the polymerization of the polymerizable species thereby forming a hydrogel at the angioplastied site.

### EXAMPLE 3

### Another Alternate Method of Treating a Diseased Vessel

A patient receives an intravenous liquid containing MV0611. After a predetermined amount of time sufficient for the MV0611 to fully distribute itself systemically in the patient's body, the patient can undergo angioplasty. The angioplasty can thereby correct problems in the stenosed vessel.

After angioplasty, eosin-Y is applied over the angioplastied lesion using a catheter. Thereafter, polymerizable species further containing tenascin are applied over the eosin-Y on the angioplastied lesion also using a catheter.

After eosin-Y, tenascin, and polymerizable species are applied at the angioplastied lesion and the MV0611 is systemically distributed though the patient's body, light at a wavelength of 532 nm can be applied to the angioplastied region. The light at 532 nm will active the MV0611 to provide photodynamic therapy at the site and will also simultaneously activate the eosin-Y which will initiate the polymerizable species thereby forming a hydrogel incorporating tenascin at the site.

### EXAMPLE 4

### Therapies Used in Conjunction with Stenting

A patient receives an intravenous liquid containing MV0611. After a predetermined amount of time sufficient for the MV0611 to fully distribute itself systemically in the patient's body, the patient can undergo angioplasty and stenting with a bare metal stent. The angioplasty can thereby expand the stenosed vessel and the stent can assist in supporting the expanded vessel.

After angioplasty and subsequent stenting, eosin-Y and polmyerizable species containing osteopontin are applied over the bare metal stent in the stented vessel. The eosin-Y and polymerizable species both migrate through the bare metal stent and adhere to the vessel wall.

After eosin-Y and polymerizable species including osteopontin are applied at the angioplastied vessel over the implanted bare metal stent and the MV0611 is systemically distributed though the patient's body, light at a wavelength of 532 nm can be applied to the region. Light at 532 nm will active the MV0611 providing photodynamic therapy at the site and will also simultaneously activate the eosin-Y which will initiate with polymerizable species thereby forming a hydrogel incorporating osteopontin at the site. Such a method will enhance vascular healing into and around the stent.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of treating a vascular medical condition comprising the steps of:
(a) selecting a surface to be treated;
(b) providing at least one photosensitive molecule;
(c) providing at least one photoinitiator;
(d) delivering said at least one photosensitive molecule to a patient;
(e) delivering said at least one photoinitiator to said surface to be treated;
(f) delivering one or more polymerizable species to said surface to be treated;
(g) providing a first light of an appropriate first wavelength to excite said photosensitive molecule at said surface to be treated, thereby forming a photodynamically treated surface; and
(h) providing a second light of an appropriate second wavelength to excite said photoinitiator at said photodynamically treated surface initiating polymerization of said polymerizable species thereby forming a hydrogel over said photodynamically treated surface.

2. The method according to claim 1 wherein said photosensitive molecule is at least one of a metal centered porphyrin molecule.

3. The method according to claim 2 wherein said photosensitive molecule is MV0611.

4. The method according to claim 1 wherein said photoinitiator is selected from the groups consisting of photosensitive dyes, quinones, hydroquinones, poly alkenes, polyaromatic compounds, ketones, unsaturated ketones, peroxides, halides, Eosin Y, Eosin B, flourone, erythrosine, flourecsein, indian yellow, derivatives thereof, and combinations thereof.

5. The method according to claim 1 wherein said surface to be treated is selected from the groups consisting of a tissue surface, a tissue implant, a medical device and combinations thereof.

6. The method according to claim 1 wherein said polymerizable species comprises a macromer formed from monomers selected from the groups consisting of methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, silicon oxides, polyethylene glycol, amide-containing monomers, isocyanide-containing monomers, derivatives and combinations thereof.

7. The method according to claim 1 wherein said polymerizable species further comprises at least one cross-linker selected from the groups consisting of N-vinylpyrrolidone, polyethylene glycol derivatives, poly vinylpyrrolidinone, poly vinylpyrrolidinone derivatives, polyamides, polyurethanes, polysulfones, acrylates, derivatives and combinations thereof.

8. The method according to claim 1 wherein said appropriate first wavelength is between about 400 nm and about 750 nm.

9. The method according to claim 1 wherein said appropriate second wavelength is between about 400 nm and about 750 nm.

10. The method according to claim 1 wherein said first appropriate wavelength and said second appropriate wavelength are the same.

11. The method according to claim 1 wherein said first appropriate wavelength and said second appropriate wavelength are provided at the same time.

12. The method according to claim 1 wherein said first appropriate wavelength and said second appropriate wavelength are provided sequentially.

13. The method according to claim 1 wherein said polymerizable species further comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, and wound healing proteins.

14. The method according to claim 13 wherein said bioactive agent comprises at least one compound selected from the group consisting of sirolimus (rapamycin), tacrolimus (FK506), everolimus (certican), temsirolimus (CCI-779) and zotarolimus (ABT-578).

15. The method according to claim 13 wherein said wound healing proteins comprise tenascin, thrombospondin and osteopontin.

16. The method according to claim 1 wherein said delivery of said at least one photosensitive molecule is by oral administration, by intravenous administration or by local delivery using a catheter.

17. The method according to claim 1 wherein said at least one photosensitive molecule and said at least one photoinitiator are the same.

18. The method according to claim 1 further comprising the step of performing angioplasty to treat said vascular medical condition.

19. The method according to claim 1 further comprising deploying a stent to treat said vascular medical condition.

20. A method of treating a vascular medical condition comprising the steps of:
(a) selecting a vessel to be treated;
(b) delivering MV0611 to a patient orally;
(c) performing angioplasty;
(d) deploying a stent into said vessel to be treated thereby forming a stented vessel;
(e) delivering said eosin-Y to said stented vessel;
(f) delivering a cross-linker and at least one macromer to said stented vessel;
(g) providing a first light of an appropriate first wavelength to said stented vessel, thereby exciting MV0611 and forming a photodynamically treated vessel;
(h) providing a second light of an appropriate second wavelength to excite said eosin-Y in said photodynamically treated vessel thereby initiating polymerization of said cross-linker and one or more macromer; and
(i) forming a hydrogel over said photodynamically treated vessel.
